(19) United States
(12) Patent Application Publication
(10) Pub. No.: US 2026/0102365 A1
(43) Pub. Date: Apr. 16, 2026

(54) Topical method for treating inflammation of skin and nasal mucosa

(71) Applicant: Edward Tak Wei, Berkeley, CA (US)

(72) Inventor: Edward Tak Wei, Berkeley, CA (US)

(21) Appl. No.: 18/445,766

(22) Filed: Jan. 25, 2024

(51) Int. Cl. A61K 31/16; A61K 9/00; A61P 29/00

(52) U.S. Cl. CPC A61K 31/16; A61K 9/0014; A61K 9/0043; A61P 29/00

(57) ABSTRACT

In screening cooling agents for their actions in the oral cavity, the molecule FEMA 4557, registered for use in chewing gum and hard candy (at <1% wt/vol), was observed to produce a long-lasting, stinging, and painful cold, like ice chips on the throat. This sensory effect of FEMA 4557, akin to noxious cold, suggested that it might have benefit for treating skin pain and inflammation. FEMA 4557 was formulated as a topical gel at ≥8% wt/vol and shown to relieve skin discomfort without causing any skin injury or irritation. The pharmacological potency of FEMA 4557 did not correlate to the EC₅₀ potency at the TRPM8 receptor. FEMA 4557 is less potent than I-menthol on this assay. The effective concentrations of FEMA 4557 ranged from ≥8 to 62% wt/vol. Surprisingly, FEMA 4557 relieved the acute inflammation from flea bites and a bumble bee sting. These FEMA 4557 formulations also relieved the inflammation of dermatological disorders such as skin allergy, urticaria, and thermal injury. The duration of the anti-inflammatory action was for several hours after a single application and skin healing was rapid. When FEMA 4557 was delivered to the inflamed nasal mucosa, it inhibited phlegm secretion in a subject with rhinitis. The conclusion is that FEMA 4557 is a potent suppressor of acute inflammation, an action not previously described for other topical agents. Thus, FEMA 4557 and related analogs may have therapeutic utility for treating inflammation.

### BACKGROUND OF THE INVENTION

### Field of Invention

One aspect of the present invention is a method to treat inflammation of the skin and nasal mucosa. The preferred embodiment, FEMA 4557, is an example of a topically applied molecule that can be used to achieve these therapeutic goals. Treatment of the skin reduces pain, itch, swelling and promotes rapid wound healing. For the nasal mucosa, treatment reduces nasal stuffiness, nasal congestion, and phlegm secretion.

### Description of the Related Art

Inflammation is the reaction of vascularized living tissue to local injury (Cotran et al., Pathologic Basis of Disease, 1989, 4^{th} Edition). The cardinal signs of inflammation were described ˜2000 years ago by Celsus and by Galen. The expression of inflamed tissues was redness (rubor), and heat (calor), due to increased blood flow, swelling (tumor) due to leakage of blood contents into tissues, and pain (dolor) due to activation of nerve endings in the injured tissue. The loss of function (functio laesa) resulted from tissue dysfunction. Much of the injury caused by inflammation is due to changes in the microcirculation, primarily an increase in vascular permeability. Leakage is due to substances released by injured tissues called inflammatory mediators, such as histamine, that enlarge gaps in postcapillary venules. This release is called the inflammatory cascade. Excess fluids and solutes entering the interstitial spaces may block gas exchange and tissue nutrition. In severe injuries, vascular leakage may persist for hours or days until thrombosis occurs, or the vessels rejuvenate. Inflammation is a dynamic process as the injured tissue seeks to defend, repair, and heal itself. In some instances, dysregulated inflammation itself becomes a source of further tissue injury, especially if the immune system is activated.

Anti-inflammatory drugs were in the clinic before mechanisms of drug action became understood. For example, non-steroidal anti-inflammatory drugs (NSAIDs), such as aspirin for pain relief, and corticosteroids, were used in rheumatoid arthritis before the cellular actions of prostaglandins and phosphorylases were known. More recent pharmacological strategies focus on therapies against tumor necrosis factors and interleukins, and inhibitors of cytokines that may be pivotal for a given local pathology. The inhibitors of tyrosine kinases in the Janus family of enzymes are also promising as a source of new anti-inflammatory drugs.

Anti-inflammatory drugs generally work as specific agonists, antagonists, enzyme inhibitors, or scavengers of inflammatory mediators. For modern drugs, each drug by design works one-on-one against substances that promote inflammation. However, the efficacy of a single antagonist may be limited if more than one mediator acts during tissue injury. For example, an antihistamine works against the histamine released in seasonal allergic rhinitis but is less effective against vasomotor rhinitis wherein histamine is one of many inflammatory mediators. An agonist, a term used to describe a chemical that activates biological events, is more effective than an antagonist if it can suppress convergent processes initiated by more than one inflammatory mediator. An example of a widely effective agonist is epinephrine for treating anaphylactic shock. Here, multiple actions of histamine are traversed by the adrenergic receptor agonist.

The treatment of acute inflammation, events that occur minutes after injury, is a matter of life and death. Witness the urgency of a heart attack or stroke, the two leading causes of death. Events occurring in the microcirculation of tissues within a brief window are critical for survival. Currently, the best treatment for the injury of skin is application of ice or cold water, wherein the treatment goal is to reduce pain and swelling (vascular leakage).

### BRIEF SUMMARY OF THE INVENTION

The applicant has investigated cooling agents, that is, chemicals that produce cooling sensations without a change in tissue temperatures. The applicant (Wei, 2012, see References for full citation) noticed that the isopropyl esters of p-menthane-glycine and p-menthane-D-alanine had prolonged cooling activity when applied to the ocular margins. Subsequently, the applicant discovered that diisopropyl analogs of phosphoryl alkanes produced refreshing coolness Yang et al. (2017). The isopropyl group appears to have specific anchoring sites on the TRPM8 receptor, which mediates sensations of coolness. Significant activity is lost if the isopropyl groups of phosphoryl alkanes change to sec-butyl. The studies of Xu et al. (2020) describe robust coupling energy between the isopropyl group on the cyclohexane ring of I-menthol and the L843 (leucine) and 1846 (isoleucine) residues of TRPM8. This “pocket” site for the “isopropyl legs” may be essential for prolonged TRPM8 receptor activation.

FEMA 3804 (WS-23), a GRAS (generally recognized as safe) food additive used as a cooling agent, has two isopropyl groups. However, when applied to the oral cavity or skin, it is short-acting with less than 30 min of cooling. FEMA 4557 (WS-27), an additive like WS-23 with two isopropyl groups, has yet to be thoroughly examined for activities on the skin or the nasal membranes. As of 2014, 30+ GRAS/FEMA cooling agents are listed (Leffingwell, 2014). One aspect of the discovery here is that the preferred embodiment, FEMA 4557, has a prolonged duration of cooling without irritancy on skin or nasal membranes, and, more surprisingly, FEMA 4557 is a potent inhibitor of acute inflammation.

The two major classes of anti-inflammatory agents, glucocorticosteroids, exemplified by cortisone, and cyclooxygenase inhibitors, exemplified by aspirin, are used to treat chronic inflammatory conditions such as rheumatoid arthritis and asthma. These drugs also have short-term anti-inflammatory effects, such as on mosquito bites and headaches. Epinephrine and antihistamines can treat the acute inflammatory effects of histamine on anaphylaxis and urticaria. However, there is no category of drugs called “acute anti-inflammatoires.” Physical lowering of temperature, for example, cooling with cold water or ice, is known to have potent acute anti-inflammatory properties. When applied to injured tissues, ice or an ice pack will stop the pain and swelling of traumatized or burned tissues. The mechanisms for how physical cooling stop acute inflammation are not known. If a chemical can mimic the effects of ice on acute inflammation, it will most likely act on a convergent point of the inflammatory cascade (Table 1). Imagine the injured tissue as a parched grassland on fire (inflamed). Dousing the field with water is a general anti-inflammatory action. Making the field wet may also dampen the chances of added conflagration.

The acute anti-inflammatory molecule identified here is FEMA 4557, a generally recognized safe (GRAS) flavoring agent approved as a food additive (Smith, 2009). Formula 1 shows the structure of the preferred embodiment, FEMA 4557.

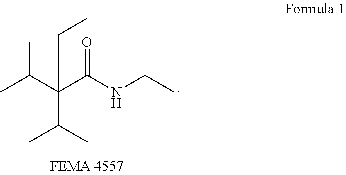

Molecules of Formula 1 are N-alkyl-diisopropyl-butanamides and first described in Roswell D G et al., U.S. Pat. No. 4,296,255, 1981. The molecules have a total mass of about 200 Daltons. Synonyms for FEMA 4557 are WS-27 and N-ethyl-2,2-diisopropyl butanamide (CAS No. 51115-70-9). The intended applications of these compositions were for toiletries (e.g., after-shave lotions and cosmetic creams), confectionery (e.g., sugar-based candy), and medicaments (e.g., antacid preparations). In the studies described here, FEMA 4557 exerts a robust anti-inflammatory activity on the skin at topical concentrations of ≥8% wt/vol. FEMA 4557 stops pain and itch and exhibits unusual healing properties on injured tissues. FEMA 4557 applied to the inflamed nasal membranes inhibits the secretion of phlegm at about 1.0 to 1% wt/vol. The effects of FEMA 4557 are not correlated to potency at the TRPM8 receptor because FEMA 4557 is a weak agonist, being less potent I-menthol in activating TRPM8. FEMA 4557 may be a prototype of a new class of anti-inflammatory agonists that affects the vascular leakage component of acute inflammation.

The vascular tree is a complex network of vessels designed to maintain, at its outermost subdivisions, a surface area between blood and tissues for exchanging gases and nutrients and for the drainage of waste products. In the immediate stages of inflammation, for example, within three hours of injury, the mechanisms regulating microvascular perfusion disrupt so that vascular patency diminishes, blood contents leak into tissues, and hemostasis may develop. The tissue interstitial fluid pressure (Pif) turns negative as hydrophilic proteins unfurl and expose hydrophilic groups to attract water. The negative Pif sucks fluids from the vascular compartment into the extracellular matrix, thus creating edema (Wiig, 2011). About three decades ago, Wei et al. (1993) and Wei and Thomas (1993) reviewed the idea of using a drug agonist to inhibit a convergent pathway for modulating acute inflammation. The hypothalamic hormone, corticotropin-releasing factor, was identified as having potent peripheral anti-inflammatory actions and was later shown to be effective for reducing peritumoral edema in human subjects with brain metastases (Robert et al., 2020). However, no lead candidates were pinpointed for topical treatment. The memory of these observations persists and has not faded. FEMA 4557 may provide new clues for the design of a peripheral anti-inflammatory medication.

In this application, the applicant accidentally found that FEMA 4557 had immediate anti-inflammatory effects. These included:
inhibition of the skin's response to insect bites (fleas and a bumble bee sting),
reducing the signs of skin irritation in a case of canine skin allergy,
reducing itch in severe urticaria, and
reducing pain and heat-induced swelling from a cigar lighter burn on the hand.
FEMA 4557 inhibited symptoms and signs of rhinitis.

The preferred embodiment, FEMA 4557, exerted an immediate, profound, and unexpected anti-inflammatory drug action not seen with related substances. FEMA 4557 rendered the immediate injury reactions of pain, irritation, burning sensations, congestion, and itch less imperative. The subject could cope with the injury. Swelling and redness went down, and healing was rapid. This rapid tissue response to local injury was unusual and occurred after topical application.

Cooling sensations are generally associated with activating the integral membrane receptor called TRPM8. However, the anti-inflammatory action of FEMA 4557 did not correlate to agonist potency at the TRPM8 receptor. FEMA 4557 is a weak TRPM8 agonist, having less potency than I-menthol. A potent TRPM8 agonist such as WS-12 did not affect inflammation of the nasal mucosa, but FEMA 4557 was active. The anti-inflammatory effect of FEMA 4557 may act on a downstream, convergent pathway of inflammation such as Pif (Table 1), but its precise molecular site of action is not yet known. If FEMA 4557 preserves tissue architecture during acute injury, it can reduce pain and accelerate healing. The dynamic events of Table 1 occur within minutes after tissue injury. The actions of FEMA 4557 suggest that it may have utility for drug treatment of acute inflammation.

TABLE 1
Inhibition of a convergent step in acute inflammation.

Tissue Injury→ release of mediators, cell membrane debris→ vascular
FEMA 4557 acts here
leakage, -Pif [symbol] edema, hypoxia→ additional cell damage, pain,
dysesthesia→ delayed effects, wound repair, immune response

### BRIEF DESCRIPTION OF THE SEVERAL VIEWS OF THE DRAWINGS

FIG. 1. Convergent nociceptive processes on skin inhibited by a cooling agent. A topical cooling gel (Intrinsic IB) applied on the skin of healthy volunteers inhibits itch induced by various pruritogens. Test pruritogens were administered into the skin with n=30 subjects per trial. Data presented by Prof. Hyeone Kim at the World Congress on Itch, Miami, Nov. 7, 2023 (the applicant is a co-author). Itch intensity AUC (area under curve) was self-rated on a scale of 0 to 10 for 10 min. Intrinsic IB (DIPA1-7 gel, Dong Wha Pharmaceuticals, Seoul, Korea) significantly inhibited itching (P≤0.01) induced by all five test pruritogens.

FIG. 2. Comparison of cooling intensity of substances tested in the oral cavity at 100 ppm in 5% sucrose. Cooling intensity was self-rated on a scale of 0 to 9 units. The data shown in this Figure is from Sonya Johnson et al., 2017. The rank order of potency of WS-5>WS-3˜I-menthol>WS-12>WS23 agrees with other compilations published by Leffingwell (2014) and by Erman (2007).

FIG. 3. Median Effective Dose (EC₅₀) of various cooling agents tested on TRPM8 in an in vitro assay. The consensus is that icilin and WS-12 are the more potent agonists, followed by WS-5>WS-3˜I-menthol. The acyclic amides WS-23 and FEMA 4557 are significantly less active than I-menthol. The applicant provided the test substances used in the publications by Mckemy et al., Bodding et al., and Sherkheli et al. Further experiments by the applicant confirmed the weak activity of FEMA 4557 on TRPM8 (10% the activity of I-menthol) and the absence of activity on TRPA1.

FIG. 4. Bioassay of FEMA 4557 (labeled as BUDA) in TRPM8 and TRPA1. Data from the laboratory of Prof. Seok-Yong Lee's at Duke University. The EC₅₀ of FEMA 4557 was estimated at 0.41 mM (left and middle panels). FEMA 4557 was not active in cells transfected with TRPA1, although a positive TRPA1 control AITC (ally isothiocyanate) was active (right panel).

FIG. 5. Cooling agents, prepared as 8% gel, were tested topically on the philtrum skin assay. The duration of cooling was recorded (hr on y-axis). The startling activity of FEMA 4557, cooling for ˜3 hr., was unexpected and surprising. FEMA 3784 is also known as Cool 10 or menthoxypropanediol.

FIG. 6. The skin's inflammatory response to flea bites was treated with topical FEMA 4557. Half an hour after the bite (left panel), the skin is swollen, and intensely itchy. After application of FEMA 4557, 32% solution, swelling and redness, is reduced (middle panel, 12 hr after bites). Surprisingly, redness, swelling, and itch significantly diminishes with a single application of FEMA 4557 and the skin heals quickly (right panel, 24 hr). Thus, a single application of FEMA 4557 is sufficient to counteract inflammation.

FIG. 7. Inhibition of pain and swelling from a bumble bee sting with FEMA 4557. The subject's distal phalanx of the middle finger was stung by a bumble bee when he tried to grab the bee with tissue paper. The volume of the distal phalanx increased from 7.0 g to 9.0 g within 30 min, as measured by the fluid displacement method over a top-loading balance. The swelling was accompanied by redness and pain. The left panel shows the normal middle finger of the contralateral hand. Half an hour after the bite (middle panel), the distal phalanx is swollen, and painful. The glistening FEMA 4557 32% solution on the finger is shown in the right panel. Surprisingly, the pain in the middle finger was converted to an intense itch within several minutes after FEMA 4557. This conversion of pain to itch is unexpected, and more tolerable to the subject. The itching lasted several hours.

FIG. 8. A male subject burned his hand with a 4-jet butane fueled cigar lighter. The burn site (white circled area in Figure) rapidly became swollen. A 20% solution of FEMA 4557 was applied with a cotton-tipped applicator to the burn site 15 min after the burn. Surprisingly the pain together with redness and swelling rapidly diminished and was not noticeable after 5 hr. FEMA 4557 was only applied once. The wound site formed an eschar which resolved within three weeks.

FIG. 9. Canine dermatitis treated with FEMA 4557. A 6-yr old long-haired dachshund had a skin allergy on the abdomen for at least eight months. Standard treatments were not effective. On the left panel, the white circle marks the inflamed sites in the right groin and belly area (with excoriations and hair loss). FEMA 4557 topically applied in 8% preparation, immediately stopped the licking, biting, and scratching of lesion sites. After three days of gel, skin healing became self-evident, and on day seven (right panel), the skin inflammation disappeared, with tissue healing and hair growth. The skin lesions have not recurred.

FIG. 10. A 53-year-old female subject developed recurrent episodes of urticaria six months after receiving a Covid-19 vaccination. Each episode of severe itching lasted ˜2 weeks, and there was 10+ episodes. The rash occurred mainly on the neck and trunk. Inspection of the nape showed evidence of scratching (lighter shade skin where the skin was abraded, white circle). Applying a 16% wt/vol FEMA 4557 gel controlled the itch, allowing the subject to rest and sleep.

FIG. 11. Quantifying inflammatory response of nasal mucosa. The secretions and exudates of the nasal cavity were measured. Subjects were instructed to blow into a pre-weighed paper tissue and record the weight of the tissue after blowing. The tissue was then inspected and marked as clear, with streaks of exudates, or with dense gel. Surprisingly, the nasal blowouts fell into three distinct weight categories that were easy to recognize. The blowout weights thus provided an objective index of nasal cavity inflammation.

FIG. 12. Inhibition of nasal secretions by topical administration of FEMA 4557, dissolved at 15 mg/mL in an ethanol, polysorbate 80, propanediol mixture, in a subject with rhinitis. Delivery into the nasal cavity was with a Nasoneb® apparatus. The test substances were rinsed into the nasal cavity for 5 sec at t=0, 2, and 5 hr. The data points were fitted by linear regression (GraphPad Prism) and the 10-hr secretion measured as (avg.±s.e.m). The results were vehicle 3.16±0.10 g, and FEMA 4557 1.86±0.07 g, with n=6, 5 observations per group, respectively. The nasal secretion-10 hr unit for FEMA 4557 was significantly lower (P≤0.01) than vehicle when analyzed by the Kruskal-Wallis test for non-parametric data.

FIG. 13. Inhibition of nasal secretions by topical administration of FEMA 4557, dissolved at 15 mg/ml in an ethanol, polysorbate 80, propanediol mixture, in a subject with rhinitis. Delivery into the nasal cavity was with a Nasoneb® apparatus. The test substances were rinsed into the nasal cavity for 5 sec at t=0, 2, and 5 hr. The data points are for individual “blow-outs” from the nose, measured with a tissue paper and balance. The average±s.e.m. for blowouts were: vehicle 0.38±0.03 g and FEMA 4557 0.21±0.03 g, with n=61, and n=56 observations per group, respectively. The blowout units were significantly different (P≤0.0001) when analyzed by the Kruskal-Wallis test for non-parametric data.

### DETAILED DESCRIPTION OF THE INVENTION

Cooling Agents from Menthol to FEMA 4557

Drug discovery usually begins with herbal remedies, but when Friedrich Serturner crystallized morphine from opium resin in 1804, modern pharmacology emerged. Now, we think of the active ingredient as a molecule of defined chemistry, not a botanical mixture. Once isolated and characterized, the active ingredient in a herbal remedy becomes a seed for a slow metamorphosis, a glacial evolution, into an improved or better entity. Witness salicylic acid in willow bark become aspirin, then ibuprofen. After Serturner crystallized morphine from opium resin, the metamorphosis spawned codeine, heroin, oxycodone, methadone, loperamide, and fentanyl.

The efforts to find better analogs from a prototype always entail extensive synthesis and bioassay, a common yardstick of synthesis being 1000+ analogs. Paul Ehrlich, the father of modern pharmacology, called his anti-syphilitic salvarsan 606 because he eventually made 900+ analogs and assays. Another example of synthetic efforts at “finding the right needle in a haystack” was Gerhard Domagk, who tested thousands of sulfonamides before finding Prontosil rubrum (which then progressed to sulfanilamide). Adams and Nicholson also screened thousands of compounds before finding ibuprofen. The prototype before ibuprofen was aspirin, which in turn is an analog of salicylic acid, first discovered as an analgesic in willow bark by Reverend Edward Stone in 1763. Drug discovery requires luck and effort. The discovery statistics are such that one would be fortunate to find ten candidates (1%) after making a thousand and refining one out of 10 (0.1%) to a lead candidate. And that lead candidate will likely fail in four out of five clinical trials!

For menthol, Wilkinson Sword (WS) scientists tried to improve menthol 50+ years ago and made 1200+ analogs. Watson et al. (1978) summarized the results in a classical paper. The WS effort ended in 1980. Leffingwell and Rowsell updated this subject in 2014; by then, thirty-two cooling agents were in the FEMA (Flavors, Extracts Manufacturing Association) database. Some WS agents are designated by FEMA as GRAS (generally recognized as safe) because FEMA has collected safety data. Collectively, a FEMA agent is periodically reviewed, and information posted online. Thus, the preference is for the FEMA label over the WS label.

The first intended use of WS analogs was for after-shave lotions (skin applications), but this shifted to cooling of the oral cavity. Today, metric tons per year of WS-3, WS-5, WS-12, and WS-23, together with menthol, are used as cooling ingredients in toothpaste (˜45%), chewing gum and confectionery (˜45%), and skin cosmetics and toiletries (˜10%). Recently, WS-5, WS-23, and FEMA 4557 (in China) entered the market as cooling substances in liquids for vaporization in e-cigarettes. However, no WS compounds ever became a therapeutic agent. FEMA 4549 and FEMA 3784 (menthoxypropanediol) were tested in clinical trials for xerotic skin but did not enter development. The “WS” designations for WS-3, WS-5, WS-12, and WS-23 are familiar from past usage. But the preferred term is now the FEMA number, which has an extensive library online.

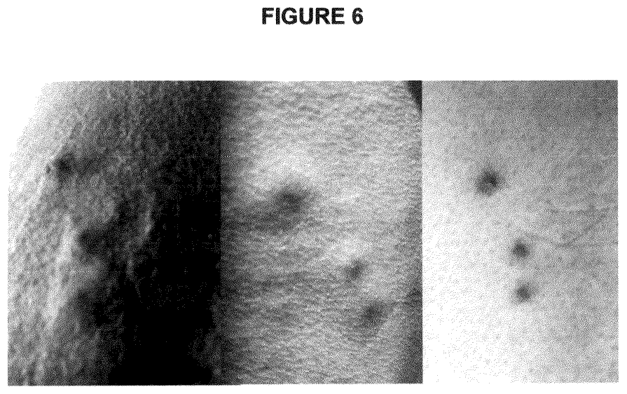

The general concept that cooling agents act on convergent inflammatory processes is illustrated in FIG. 6. A topical cooling gel (Intrinsic IB) applied on the skin of healthy volunteers inhibits itch induced by various pruritogens. Test pruritogens were administered into the skin with n=30 subjects per trial. Data presented by Prof. Hyeone Kim at the World Congress on Itch, Miami, Nov. 7, 2023 (the applicant is a co-author). Itch intensity AUC (area under curve) was self-rated on a scale of 0 to 10 for 10 min. Intrinsic IB (DIPA1-7 gel, Dong Wha Pharmaceuticals, Seoul, Korea) significantly inhibited itching (P≤0.01) induced by all five test pruritogens.

### FEMA 4557 Used for Topical Applications

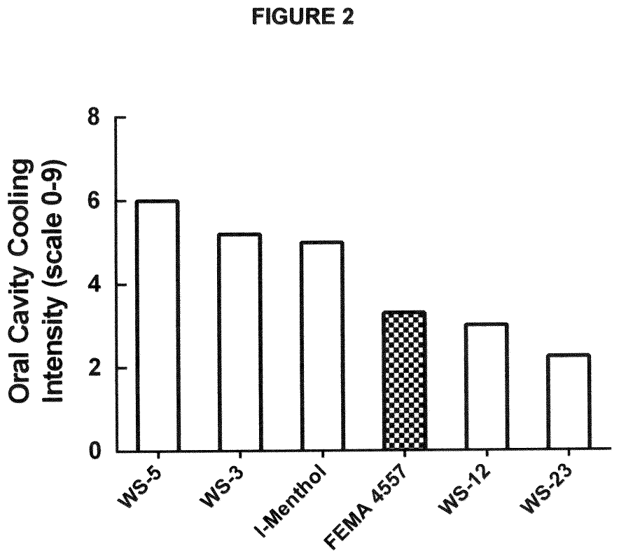

The initial screening tests conducted in this discovery were to assess candidates that produce cooling in the oral cavity. Test agents, prepared as 15 mg of white crystals, were placed on the dorsal surface of the tongue. The chemical structures and identifying characteristics are in Table 1. Surprisingly, FEMA 4557 (Formula 1, N-ethyl-2,2-diisopropylbutanamide, WS-27) stood out as a potent agent producing stinging, freezing, and painful cold on the throat (Table 3). Related cooling agents were not freezing cold, except for WS-23, but this analog was too short-lived for practical use. FIG. 2 shows the relative cooling intensity of various FEMA compounds, as reported by Leffingwell (2014) and Johnson et al. (2018), but which differ from Table 3.

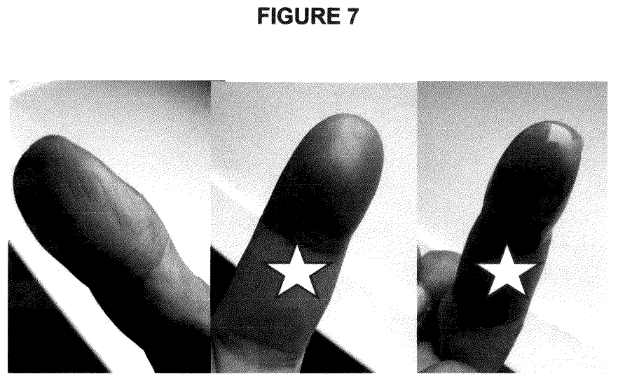

After the oral cavity screen, FEMA 4557 was formulated into a topical gel and applied to the skin surface. This FEMA 4557-gel effectively treated skin dysesthesia in subjects with insect bites, skin allergies, and thermal injury (FIGS. 7 to 10, vide infra). Related FEMA agents, such as FEMA 3804 (WS-23), WS-5, and WS-12, were ineffective under similar test conditions. The doses for FEMA 4557's topical activity on the skin were much higher than doses used commercially in the oral cavity. However, no tissue irritation or inflammation appeared on the skin, despite concentrations of >5% wt/vol. The average use levels (ppm)/average maximum use levels (ppm) for FEMA 4557, at which the FEMA Expert Panel based its judgments that the substances are generally recognized as safe (GRAS), is 3000/6000 ppm (or 0.3 to 0.6%) for FEMA 4557 in chewing gum, and 1000/1500 for confectionery frostings and hard candy. The skin tests confirmed and revealed the therapeutic value of FEMA 4557 as a topical medication.

An example of a topical gel formulation of FEMA 4557 consisted of 8 to 16% wt/vol of FEMA 4557, 2% ethanol (or 1,2-propanediol) as a solvent, and 0.5% carbomer 980 as a gelling agent. L-Menthol may be included as a dispersing agent, penetration enhancer, and as an initiator. When applied topically, this gel will stop the irritation of an insect sting for 3+ hours with an onset of less than 3 minutes. In summary, a known flavoring agent, used primarily as an agent in the oral cavity, was found to be an effective pharmaceutical ingredient for topical application to treat acute inflammation.

### Chemistry of Cooling Agents and Terminology

The prototype cooling agent is I-menthol, which occurs in plant species of the Mentha family. L-Menthol is one of eight enantiomers and has the greatest cooling activity. The WS compounds most widely used in commerce (WS-3, WS-5, and WS-12) are p-menthane carboxamides. p-Menthane has a compact cyclohexane ring with the steric configuration of I-menthol. WS scientists also discovered “acyclic amides” that exerted cooling actions (Rowsell et al., 1981). The acyclic amide, WS-23 and WS-27 were commercialized in China for liquids used in electronic cigarettes (Wu et al., 2020; Yogeswaran et al., 2022). These compounds are “acyclic” because they do not contain the cyclohexane ring. The applicant described the properties of the cooling agents icilin and 1-dialkylphosphorylalkanes, represented by 1-diisopropylphosphorylheptane (synonyms: Cryosim-1, DIPA-1-7: CAS No.) which do not contain cyclohexane.

The monoterpenoid sensates (e.g., menthol, eucalyptol, camphor), p-menthane carboxamides, dialkylphosphorylalkanes, icilin analogs, and acyclic amides can generate an infinite variety of analogs. However, which will work in the clinic? In practice, one cannot test thousands of compounds for clinical utility. The number of lead candidates must be sifted and manageable.

In searching for lead candidates, the usual idea is to find the most potent analog, i.e., the one with lowest EC₅₀ at the putative receptor. But this approach would have led to a dead end. Instead, structural features were identified that might provide better clues on bioactivity. An unusual lead candidate was identified and by chance it was found to have profound anti-inflammatory activity in vivo.

### Structure-Activity and Isopropyl Legs

In previous studies, Wei (2012) found that the isopropyl esters of p-menthane carboxamides had prolonged cooling activity on ocular margins, by contrast to methyl, ethyl, and butyl esters. Subsequently, he described 1-diisopropylalkylphosphorylalkanes as potent stimulators of the TRPM8 receptor. These molecules, called “Cryosims,” are effective for urticarial rashes and scalp dermatitis in human subjects (Jung et al., 2021; Kang et al., 2022). FEMA 4557 and FEMA 3804 (WS-23) are similar structures (Table 2). Both have prominent isopropyl legs in close juxtaposition to the hydrogen-bonding oxygen atom. FEMA 4557 produced a potent stinging cold on the tongue, but not FEMA 3804 (Table 3). The extra methyl group (marked as *** in Table 2) of FEMA 4557 may fit better into the receptor pocket. Increasing the N-alkyl substituent to n-propyl, isopropyl; or acetyl may further prolong the duration of action.

Xu et al. (2020) conducted a thorough analysis of the binding of I-menthol to the mouse TRPM8 receptor. They noted the robust coupling energy between the isopropyl group on the cyclohexane ring and the channel based on the Kd values. For the critical binding amino acid residues, they stated that the highest coupling energy values for the “isopropyl leg” were at L843 (leucine) and I846 (isoleucine). In contrast, the V775 (valine) residue outside the binding pocket had smaller coupling energy with the isopropyl legs. Y745 (tyrosine) and F839 (phenylalanine) were within the binding pocket, but they exhibited smaller coupling energy than 1846 and L843. Together, these results suggested that the isopropyl legs of the more potent agents point downward and stand on residues L843 and I846 to facilitate channel activation.

TABLE 2
Chemical structures of FEMA 3804 and FEMA 4557
FEMA 3804 (WS-23)

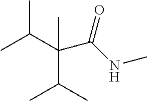

FEMA 4557 (WS-27)

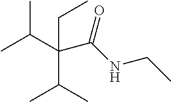

***(hook) on FEMA 4557
FEMA 4557

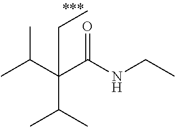

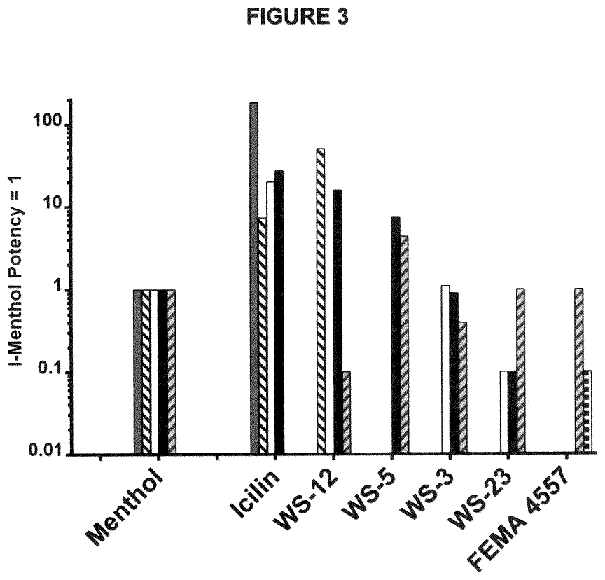

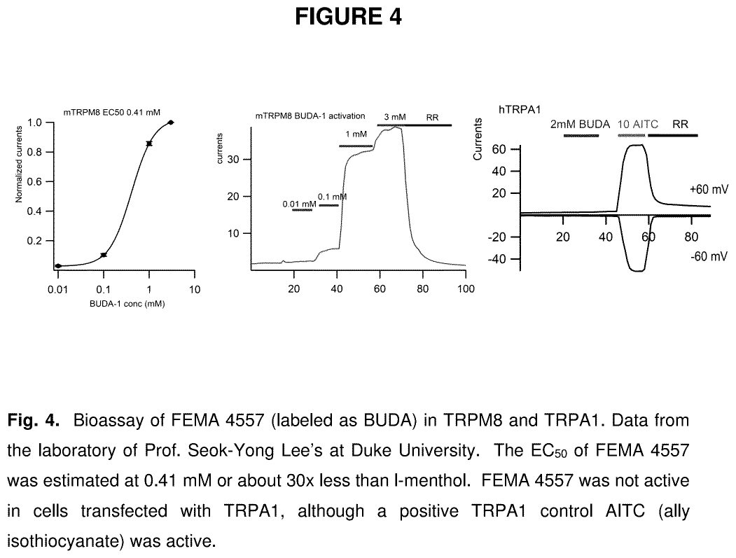

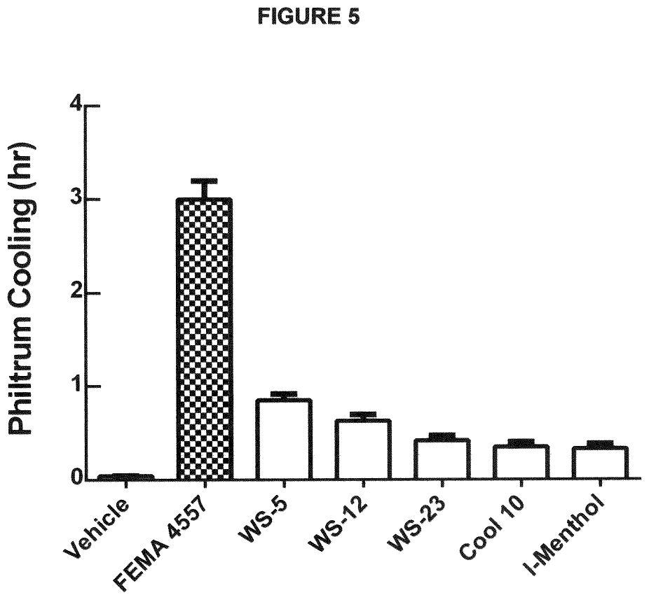

The diisopropyl “legs” of FEMA 4557 made it attractive for further screening. Most data on structure-activity relationships of p-menthane carboxamides are for cooling activity in the oral cavity (FIG. 2) and for TRPM8 receptor activation (FIG. 3, EC50 values). However, we find that FEMA 4557 had low potency on TRPM8 (FIG. 4) but an unexpected and surprisingly long duration of cooling action when applied to the philtrum skin (FIG. 5). The extra methyl group on FEMA 4557, marked as (***), and which is not on WS-23, may act as a “hook” or “coat-hanger” to trap the molecule at the receptor pocket and prolong occupancy and duration of action (Table 2). FIG. 5 shows the relative activity of FEMA 4557 to other compounds. FEMA 4557's potency on the philtrum test is exceptional: it is 3.5× more potent than WS-5 and 7.2× more potent than WS-23. This potency is not correlated to TRPM8 potency, wherein FEMA 4557 is less potent than I-menthol and the other analogs (Table 4). FEMA 4557 is active on the philtrum at 64% wt/vol, which shows its unusual solubility characteristics.

TABLE 3
Isopropyl legs in the structure activity analysis of agents with cooling
activities in the oral cavity.

| FEMA | | | | Effects of 5 to 15 |
|---|---|---|---|---|
| No. | CAS No. | Synonyms | Structure | mg on tongue |
| 2665 | 2216-51-5 | I-Menthol | 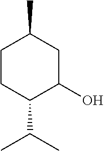 | bitter taste, aroma of menthol, pleasant mild cooling |
| — | 1487170- 15-9 | Cryosim-1 | 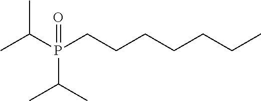 | stinging, painful cold; only 5 mg tested |
| | 1401555- 39-2 | Ax-8 p-Menthane-Gly-OiPr Wei U.S. Pat. No. 8,426,463. | 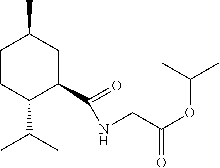 [SMILES: CC(C)OC(=O)CNC(=O)[C@@H]1C[C@H](C)CC[C@H]1C(C)C] | pleasant cooling in oral cavity, ~30 min, no freezing cold or pain, or sting |
| 4557 | 51115-70-9 | WS-27, N-ethyl-2,2- diisopropyl butanamide, N,2-diethyl-3-methyl-2- (1-methylethyl)- butanamide | 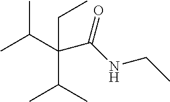 | stinging, painful cold ~35 min |
| 3804 | 51115-67-4 | WS-23 | 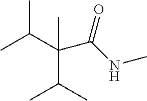 | robust cooling, bitter taste, ~18 min |

TRPM8 Receptor Potency, the EC₅₀ In 2002, D. Julius and A. Patapoutian (Nobel Laureates, 2021) showed that menthol and icilin, both cooling agents, acted on a neuronat membrane protein later called the TRPM8 receptor. A receptor assay is a standard rapid screening tool. For example, in a 384-plate assay, 10 compounds are tested to yield dose-response data. The in vitro TRPM8 assay generates an EC₅₀ (median effective concentration) to indicate potency. However, quantitative correlations between the EC₅₀ on TRPM8 and cooling sensation is not exact. The science is “roughly” predictive, but each agonist has considerable uncertainty and variability on the chosen target surface. The EC₅₀ has scientific panache but do not inform on duration of action or quality of sensation. Table 4 summarize TRPM8 EC₅₀ (see FIG. 3) from various groups. The consensus is that icilin and WS-12 are the more potent agonists, followed by WS-5>WS-3 ˜I-menthol. The acyclic amides WS-23 and FEMA 4557 are significantly less active than I-menthol. The applicant provided the test substances used in the publications by McKemy et al., Bodding et al., and Sherkheli et al. Further experiments by the applicant confirmed the weak activity of FEMA 4557 on TRPM8 (10% the activity of I-menthol) and the absence of activity on TRPA1 (FIG. 4).

TABLE 4
EC50 of Cooling Agents Tested on TRPM8, See FIG. 3 for graph

| | EC50 | | EC50 | | EC50 | | EC50 | | EC50 |
|---|---|---|---|---|---|---|---|---|---|
| Ref#5 | μM | Ref#1 | μM | Ref#2 | μM | Ref#6 | μM | Ref#3 | μM |
| icilin | 0.36 | Icilin | 0.2 | WS-12 | 0.2 | Icilin | 7 | WS-5 | 0.5 |
| menthol | 67 | WS-3 | 3.7 | CPS-113 | 1.2 | WS-12 | 12 | FEMA- | 1.6 |
| | | | | | | | | 4557 | |
| | | menthol | 4.1 | Icilin | 1.4 | WS-5 | 26 | menthol | 2.2 |
| | | Cool 10 | 6.0 | CPS-369 | 3.6 | CPS- | 65 | WS-23 | 2.2 |
| | | | | | | 369 | | | |
| | | [symbol] | [symbol] | WS-148 | 4.1 | menthol | 196 | WS-3 | 5.9 |
| | | | | WS-30 | 5.6 | WS-3 | 210 | WS-12 | 22.1 |
| | | | | menthol | 10.4 | WS-23 | 1500 | WS-24 | 34.5 |

Reference # in Table 4 and plotted in FIG. 3
1. Behrendt, H-J. et al., Brit. J. Pharmacol. (2004) 1-9..
2. Bödding M, et al.. Cell Calcium. 2007; 42(6): 618-28.
3. Johnson S, et al., J Agric Food Chem. 2018; 66(10): 2319-23.
4. Leffingwell J, Rowsell D. Wilkinson Sword cooling compounds: from the beginning to now. Perfume & Flavorist 39: 34-44, 2014. Provides a similar analysis of published data.
5. McKerny D D et al.. Nature. 2002; 416(6876): 52-8.
6. Sherkheli MA, et al., J. Pharmacy and Pharmaceutical Sciences 2010; 13(2): 242-53.

The results of Johnson et al. (Ref #3) differ most from the data of others. Inspection of Johnson's data shows the numbers do not fit the Hill Equation criteria for an EC₅₀ measurement. In a Sigmoid Curve, it is essential to have data points above and below the median value, but Johnson's data does not have it. Overall, one can conclude that the acyclic amide coolants, WS-23 and FEMA 4557, are less potent (e.g., ⅕ to 1/10) than menthol. The result on FEMA 4557 was from Prof Lee's laboratory at Duke University (Prof Lee is the world's foremost expert on TRPM8, having published three papers in “Science” on this subject in the past four years.) Thus, the surprising potency of FEMA 4557 on the philtrum test is unexpected (FIG. 5). The bioassay data of FEMA 4557 (labeled as BUDA) in TRPM8 and TRPA1, were generated in the laboratory of Prof. Seok-Yong Lee at Duke University. The EC₅₀ of FEMA 4557 is at 0.41 mM or about 1/30× that of I-menthol. FEMA 4557 was not active in cells transfected with TRPA1. A positive TRPA1 control AITC (allyl isothiocyanate) was active on TRPA1.

See FIG. 2. Cooling intensity of agents tested in the oral cavity at 100 ppm in 5% sucrose. Cooling intensity was self-rated on a scale of 0 to 9 units. Data are from of Sonya Johnson et al., 2017. The rank order of potency of WS-5>WS-3˜I-menthol>WS-12>WS23 agrees with compilations published by Leffingwell (2014) and by Erman (2007).

The sensory neurons of the skin, studied in isolated dorsal root ganglia neurons, have TRPM8 as the primary protein for detecting cold. However, in about 20% of such labeled neurons, the TRPM8 co-exists with TRPV1 in the same neuron. Co-activation of such TRPM8/TRPV1 neurons may give the sensation of icy, stinging, painful cold. TRPA1 is another TRP channel that co-exists with TRPM8 and TRPV1. Johnson et al., 2017 reported that FEMA 4557 activates TRPA1, but we cannot confirm these results. The precise target receptor of FEMA 4557 for anti-inflammatory activity requires further study.

### Philtrum Skin Test for Quantifying Topical Bioactivity

John Gaddum, one of the pioneers of modern pharmacology, helped establish the concept of Bioassay and the EC50. He was fond of pithy sayings. Some memorable quotes are:
A branch of science comes of age when it becomes quantitative.
On research. “Enthusiasm is not enough.”
“The pharmacologist has been a ‘jack of all trades’, borrowing from physiology, biochemistry, pathology, microbiology, and statistics—but he has developed one technique of his own and that is the technique of bioassay.”

In the search for a molecule that will suppress inflammation after topical application, it is essential to have the right tests, even if the circumstances of injury are by accident. Wilkinson Sword in their program for finding better menthol analogs, tested substances by impregnating them on filter paper and applying the paper to the tongue of volunteers. The threshold for cooling on the tongue surface was rated with “+” signs to signify potency and later quantified as “μg” on the tongue. The data were “semi-quantitative” and there is overlap in the potency of many analogs. As WS compounds became widely used in the 1990s in toothpaste, chewing gum, mouthwash, and confectionery, there was a need to re-design the bioassay for the oral cavity. The WS method was replaced by the “hold and spit” method. In this assay, a test substance is suspended or dissolved in a liquid, and the liquid held for 30 sec in the mouth and then expectorated. Sometimes, sucrose is added to the test liquid. The sensory effects are recorded by the subject and “cooling intensity in the oral cavity” is rated on a scale of 0 to 9. No attempt was made to relate oral cavity cooling to effects on skin. The general consensus was that WS-5 gave the most intense cool, followed by WS-3 and I-menthol, and then WS-12. WS-23 and WS-24 were considered less potent.

In 2002, groups led by David Julius and Ardem Patapoutian (Nobel Laureates of 2021) showed that menthol and icilin, both purported cooling agents, acted on a neuronal membrane protein called the TRPM8 receptor. A TRPM8 receptor assay is now a standard tool for assessing cooling potency, and can be done quickly. For example, in a 384-plate assay, 10 compounds can be tested for dose-response data. A chemical agonist, an in vitro TRPM8 assay generates an EC₅₀ (median effective concentration). But the correlation between the EC₅₀ and cooling is not always clear. That is, the science was “roughly” predictive, but there was considerable uncertainty for each agonist. The EC₅₀ values have scientific panache, but do not give information on the duration of action or the quality of the sensation.

Wei proposed the use of the skin on the philtrum as a test site for cooling activity of chemicals. The philtrum is the midline groove above the upper lip border and vermilion and is the drug application site. The gel is placed on the philtrum with a cotton-tipped applicator up to the nasolabial folds and sometimes, but not deliberately, on the lower nostrils (subnasale). Cold receptors densely innervate the philtrum skin, second only to the surfaces of the eyeball and anogenitalia. The lips also have a high density of “cold spots.” This is called the philtrum assay. The test substance is dissolved in a carrier, such as solvent, gel, or ointment and applied to the philtrum, a surface area of ˜10 to 12 cm2. The subject records sensations on the philtrum and environs on a scale of 0 to 3, as: 0=no changes in sensation, 1=a slight feeling of coolness, cold or tingling, 2=a clearcut feeling of coolness or cold and 3=a robust feeling of coolness and cold. The intervals for measurements are usually 0, 1, 3, 5 min after application, and at then 5 min or longer intervals thereafter until two successive zeros are obtained. Test Substances Applied to the Philtrum. Dip a cotton-tipped applicator (Puritan Cotton-tipped applicators, or Q-Tips) into a reservoir bottle containing the liquid or semi-liquid (gel) test substance. The composition adhering to the cotton (about 0.2 mL) is put on the skin. A subject can practice the method in front of a mirror with a high-intensity light source.

TABLE 5
Comparison of acyclic amide to p-menthane carboxamide cooling agents in the
oral cavity. The FEMA designation means an Expert Panel considers the compounds as
generally recognized as safe (GRAS) for its intended use.

| FEMA | CAS | | | Effects of 15 mg |
|---|---|---|---|---|
| No. | No. | Synonyms | Structure | on tongue |
| 3804 | 51115- 67-4 | WS-23, N- methyl-2,2- diisopropyl propanamide | 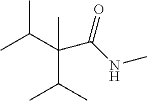 | painful cold in throat, ~18 min |
| 4557 | 51115- 70-9 | WS-27, N- ethyl-2,2- diisopropyl butanamide | 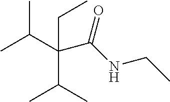 | stinging, painful freezing cold in throat, >35 min |
| 3455 | 39711- 79-0 | WS-3 FEMA 3455 | 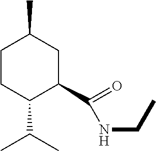 | pleasant cooling in oral cavity, ~8 min, no freezing cold or pain, or sting |
| 4309 | 68489- 14-5 | WS-5 FEMA 4309 | 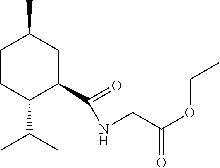 [SMILES: CCOC(=O)CNC(=O)[C@@H]1C[C@H](C)CC[C@H]1C(C)C] | pleasant cooling in oral cavity, ~12 min, no freezing cold or pain, or sting |
| 4681 | 68489- 09-8 | WS-12 FEMA 4681 | 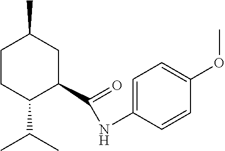 [SMILES: COc1ccc(NC(=O)[C@@H]2C[C@H](C)CC[C@H]2C(C)C)cc1] | pleasant cooling in oral cavity, ~20 min, no freezing cold or pain, or sting |

TABLE 6
Results with Test Substances, see FIG. 5 graph.

| | % Conc. | Duration | |
|---|---|---|---|
| Chemical | wt/vol | (min) | Comments |
| FEMA | 8 | 180+ | icy cold sensations, prolonged |
| 4557 | | | cooling, anti-itch effects on flea bites |
| | | | and other skin irritations such as |
| | | | pollen allgergies, qualitatively and |
| | | | quantitatively different from the other |
| | | | test analogs, no irritation of the skin |
| | | | was observed with FEMA 4557 and |
| | | | concentrations of up to 12% weight/ |
| | | | volume applied to the philtrum skin. |
| | | | The average time of cooling for 2, 4 |
| | | | and 8% concentrations were 50, 95 |
| | | | and 175+ min, respectively. |

Other compounds tested on the philtrum assay at 8%.

| | | | |
|---|---|---|---|
| WS-23 | 8 | 25 ± 3 | short duration of action, no overt |
| | | | cold, but more coolness |
| WS-12 | 8 | 38 ± 4 | pleasant cool sensations on skin |
| WS-5 | 8 | 51 ± 4 | pleasant cool sensations of skin |

FIG. 5. Cooling agents, prepared as an 8% wt./vol. gel, was tested topically on the philtrum skin assay. The duration of cooling was recorded (min on y-axis). The startling activity of FEMA 4557, cooling for ˜3 hr, was unexpected and surprising. FEMA 3784 is also known as Cool 10 or menthoxypropanediol.

Note that where R=ethyl, the compound Is FEMA 4557 and has been described in Roswell et al., U.S. Pat. No. 4,296,255, 1981. The isopropyl, n-propyl cyclopropyl, or acetyl analogs have not been synthesized and tested. There is reason to believe that these analogs may be more potent than the ethyl analog. The extra methyl group forming the “hook” or “coat hanger” on FEMA 4557 is critical for the practice of this invention. The hook is ethyl in FEMA 4557, but can be modified to n-propyl, or i-propyl to increase activity, as shown in Formula 2. Permeation through the stratum corneum is not an essential factor for success because FEMA 4557 is much more active than WS-23, in spite of having an extra methyl group. Sun (2006) has described N-alkoxyalkyl-2,2-diisopropyl-propanamides with lower melting points that FEMA 3804 (WS-23).

TABLE 7
Structure of FEMA 4557 Analogs Contemplated in this Discovery

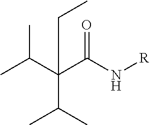

Formula 1
wherein R is ethyl, isopropyl, n-propyl, cyclopropyl, or acetyl

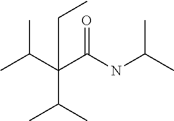

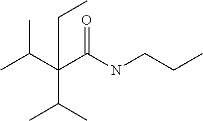

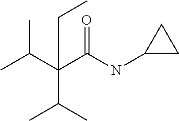

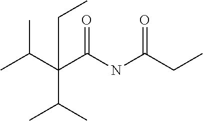 [SMILES: CCC(=O)NC(=O)C(CC)(C(C)C)C(C)C]

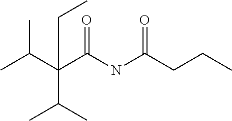 [SMILES: CCCC(=O)NC(=O)C(CC)(C(C)C)C(C)C]

### Formulation, Excipients, and Initiators

The formulation of skin care products is a secretive process. Success may depend on slight changes in the selection or levels of ingredients without an obvious explanation for the differences. Here, the choice of the carbomer polymer for gelling is straightforward, and varying the concentration will determine the appropriate gel viscosity.

The dispersion of FEMA 4557, the active ingredient, is facilitated by ethanol and, surprisingly, by about an added 1% by weight of I-menthol. The menthol helps FEMA 4557 to be homogeneous and dispersed in the gel. Ethanol, as a solvent, must be used at a minimal concentration because it inhibits TRPM8 agonist receptor binding. By using an ethanol concentration of <1% wt/vol helps reduce this interference.

Another ingredient for the formulation is an initiator of cooling. An 8% wt/vol gel of FEMA 4557 alone has an onset of 15 to 20 min for producing cool and cold. An initiator added to the formulation immediately makes the cold within 1 to 3 min and sustains the anti-itch effect. The initiator enhances the patient's appreciation of the anti-itch impact. Wei in U.S. Pat. No. 10,195,217 (2019) describes the chemistry of some initiators. L-Menthol is an example of an initiator and a penetration enhancer on the skin. The use of initiators is part of the discovery process. Other compounds to help dissolve or extend the duration of action, such as polysorbate 80, polyethylene glycol, propanediols, and hyaluronic acid, may be considered as excipients.

Injury to tissues happens by accident. As the applicant was working with FEMA 4557 compositions, he encountered flea bites, a bee sting, thermal injury from a cigar lighter, a case of canine skin allergy, and cases of urticaria. The surprising finding is that single applications of FEMA 4557 had a profound anti-inflammatory effect on the cardinal signs of the inflammation of these injuries. The anti-inflammatory effects of FEMA 4557 are unusual, unique for its chemical class, and not previously described.

### Study 1, Insect Bites (Fleas and a Bumble Bee)

An elderly male gardener developed an intense itch on his thigh. The row of inflamed spots is typical of flea bites (FIG. 6). Half an hour after the bites, the skin is red; swollen, and intensely itchy (left panel). Within 10 min after application of FEMA 4557, 32% solution, using a pen applicator, swelling, and redness were reduced (middle panel at 12 hr). Surprisingly, a single application of FEMA 4557 is sufficient to suppress the skin dysesthesia (irritation, itch, burning sensations and pain). After 24 hr, the skin inflammation heals. The rapid onset of the effectiveness of a single application of FEMA 4557 to counteract the flea bite inflammation is surprising and unexpected. Subsequently, the ability of FEMA 4557 to counteract the inflammation of insect bites (mosquitoes and fleas) was seen in 12 more cases.

A furry yellow bumble bee, about 1.5 cm long, was buzzing behind the windowpane in the kitchen. Instead of swatting the bee, the applicant tried to trap the bee with a piece of tissue. He was stung on the distal phalanx of the middle finger (FIG. 7), which became swollen and sharply painful. The volume of the distal phalanx increased from 7.0 g to 9.0 g within 30 min, as measured by the fluid displacement method over a top-loading balance (middle panel). A 32% wt/vol FEMA 4557 solution glistens on the fingertip (right panel). Surprisingly, the sharp pain transforms into an intense itch within a few minutes. This itch persisted for at least three hours.

The principal pain-inducing substance in bee sting venoms is a 26-residue peptide called melittin (Chen et al., 2016). Melittin affects membrane pores, has multiple actions of various nociceptive receptors, such as TRPV1, and initiates cascades of phospholipid inflammatory mediators. The ability of FEMA 4557 to convert a painful melittin sting to itch may offer clues on the treatment of acute pain. If acute pain from tissue injury converts to an itch after FEMA 4557, the itch is more tolerable than pain.

### Study 2, Thermal Injury

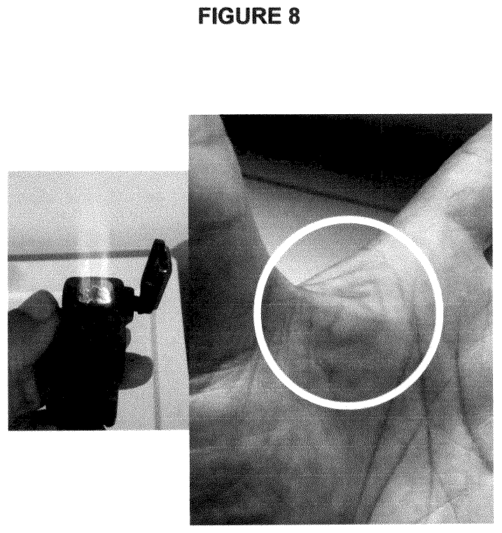

FIG. 8 shows a 4-jet butane-fueled cigar lighter with a cigar punch. The cigar punch on the side of the lighter makes a hole on top of the cigar to facilitate airflow during smoking. When lit, the lighter's flame is like a blowtorch. A subject burned his hand when trying to use the cigar punch. FIG. 8 shows the burn site from the cigar lighter at the juncture of the forefinger and thumb of the left hand (white circled area). There was pain after the burn, together with redness and swelling. A 20% solution of FEMA 4557 was applied to the site with a cotton-tipped applicator 15 minutes after the burn. The pain diminished and was not noticeable after 5 hr. FEMA 4557 was applied once. The redness, swelling, and pain disappeared after 24 hours, and the wound site formed an eschar, which resolved within three weeks. The rapid suppression of pain and healing without side effects was surprising and indicated a potent anti-inflammatory effect on thermal injury.

### Study 3. Canine Skin Allergy

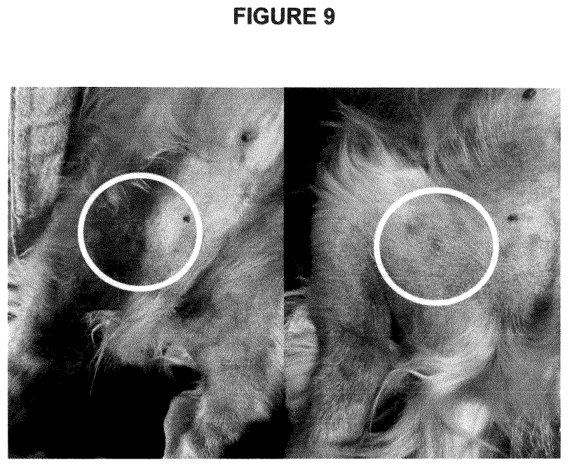

A six-year-old long-haired Dachshund developed an inflamed area on the belly and lower right groin over eight months (FIG. 9). Upon examination, the lesions were red and swollen, without hair, and excoriated (white bar). The dog frequently licked and tried to scratch these sites. Veterinarian visits ruled out fleas, mites, fungi, and bacteria growth. The condition was diagnosed as “a non-specific allergy.” Some recommended procedures, oral intake of Zyrtec, an antihistamine, anti-fungal shampoos containing—conazoles, topical steroids, and changing the diet were not effective.

The skin inflammation persisted. The owner was given a dispenser with a reservoir containing 8% wt/vol of FEMA 4557 gel dispersed in 4 mg/ml of polyacrylic gel (Lubrizol, Carbopol 980), and formulated with 1% ethanol and 1% menthol. A cotton-tipped application was used to apply the gel to the lesion sites. Surprisingly, scratching and licking diminished within one day after application, and after three days of daily application, it was evident that the gel was reducing the redness and swelling. The gel was given for one week. The lesion sites healed, with the excoriation sites replaced by scabs (FIG. 9). The hair grew back over the injured skin. Surprisingly, the skin allergy did not recur in the six months after this treatment.

### Study 4. Urticaria and Histamine Release

Urtica (Latin for sting) is a genus of flowering plants from the family Urticaceae. Many species have stinging nettles that contain histamine, serotonin, and acetylcholine. The mediators account for the signs and symptoms of urticaria, a human skin condition of histamine release triggered by an IgE-antigen antibody type of allergy.

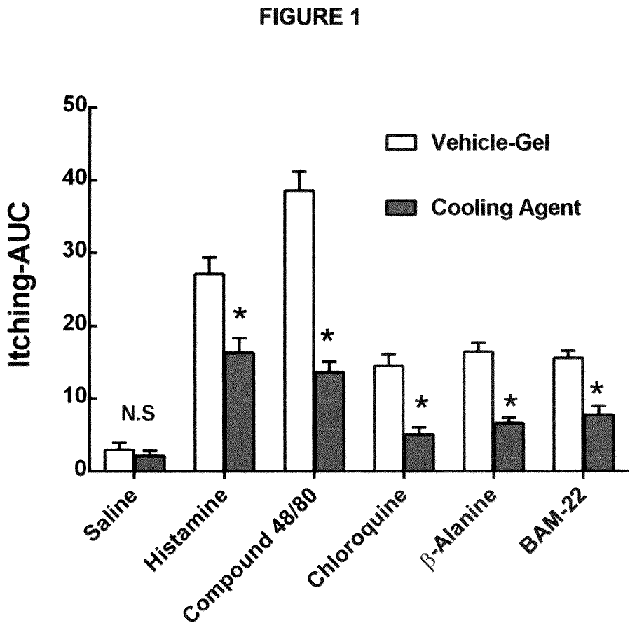

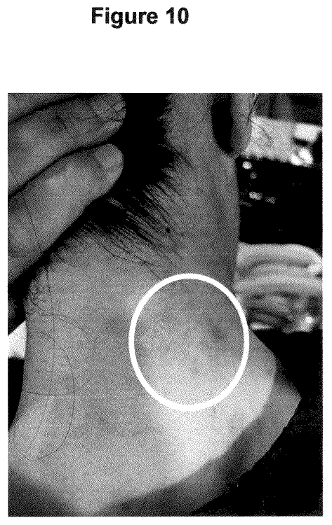

A 53-year-old female subject developed recurrent episodes of urticaria six months after a COVID-19 vaccination (FIG. 10). The itch of urticaria was on all parts of the body. Each episode lasted ˜2 weeks, and there were 10+ episodes in the post-vaccination period of one year. FIG. 10 shows the urticarial lesions on the nape. There is evidence of scratching (lighter skin where the epithelium was abraded) and rash (darker skin). Applying FEMA 4557 gel or solution controlled the itching within 10 min, allowing the subject to rest and sleep. The subject is now controlling her skin lesions with daily doses of fexofenadine (Allegra/Hives) and using the FEMA 4557 gel on an “as-needed basis.” As shown in FIG. 1, cooling agents suppress the itch of many pruritogens on the human skin.

### Study 5. Skin Dysesthesia, Itch. and Inflammation

The compositions and formulations contemplated here have special utility in skin inflammation of the mature patient and the patient with the genitourinary syndrome of menopause. Everybody gets old, and all females undergo menopause, so this population is large.

The terminology to describe aging and pruritus has evolved from “senile pruritus” to the itch of the elderly and now the itch of the mature patient. The exact age of being elderly is between sixty to sixty-five years, but the risks of itch progress, so about 50% of individuals over eighty have itch. The prevalence was measured in surveys of the elderly in nursing homes. The exact causes of itching in the mature patient are varied and often unknown, but include psychogenic and neurogenic triggers, inflammation, xerosis, and allergy.

Intense physical cooling with ice suppresses skin inflammation and dysesthesia, but this is not sustainable because the ice will damage tissue metabolism. A potent topical glucocorticoid has anti-inflammatory actions, but its onset is slower and will not work for thermal injury. A potent cooling agent such as Cryosim-1 (also known as DIPA-1-7, 1-diisopropylphosphorylheptane) is antipruritic and analgesic. However, it does not affect the redness and the diameter of the wheal produced by histamine, so it is not anti-inflammatory.

The therapeutic indications and treatment goals for FEMA 4557 are to treat the skin dysesthesia of the mature patient, the patient with diabetic pruritus, and vulvar itch. These skin sites are usually keratinized but may become more permeable because of inflammation and scratching. The number of epithelial cell layers is ˜8 to 15. The clinical conditions are chronic and not “curable.” The treatment goals are to ameliorate symptoms and interrupt the scratching cycle. A composition containing the preferred embodiments has the advantage of immediate onset of drug action.

TABLE 8
Therapeutic Indications for FEMA 4557 on Skin Dyesethesia

| Indication | Population | Sites of Inflammation | Quality of Life |
|---|---|---|---|
| pruritus of | ~10%+ of world's | extremities and torso | Persistent, |
| diabetes | population has | | disruptive and |
| | diabetes | | aggravating |
| pruritus of | onset >60 to 65 | extremities, torso, | Persistent and |
| mature | years, severe | scratching increases | disruptive |
| patient | at >80 years | risks of infection | |
| vulvar | Post-menopausal | genitourinary system | Persistent and |
| pruritus | women | | disruptive |

The preferred embodiments, compositions, and formulations contemplated here have special utility in the treatment of dermatitis of the patient with diabetes, the mature patient, and inflammation of the genitourinary system (Table 8). Currently, the therapeutic options for these conditions are limited.

Dermatitis and Itch in Patient with Type 2 Diabetes (T2DM). T2DM occurs in ≥10% of the world's population and individuals become more overweight and have a sedentary lifestyle. T2DM is readily defined by lab tests on fasting plasma glucose levels (≥126 mg/mL) or elevated glycated hemoglobin. The diabetic state is associated with dry skin (xerosis) and neuropathy and such T2DM patients have chronic dermatitis (˜25%) which disrupts the quality of life. Obesity and diabetes also contribute to chronic kidney disease which contribute to skin inflammation. A preferred embodiment which reduces dermatitis and skin dysesthesia will have benefit in such patients.

Skin Dysesthesia of the Mature Patient. Everybody gets older. The skin, the body's largest organ, deteriorates with age. The skin becomes xerotic and is more fragile, and there are defects in wound healing. Dermatitis is a prominent symptom seen in geriatric dermatology. The medical terms to describe aging and dermatitis have evolved from senile pruritus to “itch of the elderly” and now “itch of the mature patient.” The age for being “elderly or mature” varies from 60 to 65 years, but at about 80 years, 30% of individuals are likely to have a chronic itch. “Chronic” means the itch is present for more than six weeks. The itch of the mature patient is a serious management problem in nursing homes. The causes of itching in older patients are varied and often challenging to diagnose. Triggers for itch include cutaneous defects and non-cutaneous causes such as psychogenic and neurogenic factors, medications, and allergies. The exact causes of itching in the mature patient are often not known, Treatments include creams and emollients, anti-inflammatory steroids, antihistamines, and psychotropic drugs. A topical gel, lotion, or cream to reduce itching in the mature patient would be valuable to physicians and patient.

An excellent illustration of diabetic dermatitis is shown in Murao et al. (2021). Reviews of the topics of “itch of the mature patient”, incorporated herein by reference are Chung B Y et al. (2021); Shevchenko A et al. (2021). Kahremany et al. (2020) provides a general background on skin dysesthesia.

The three clinical conditions have these features in common:
The dermatitis is a dominant symptom that destroys the QoL for the patients.
The three indications are not “curable” but chronic and persistent.
The philtrum assay is valuable to discover active ingredients and optimal formulation.
The dermatitis occurs on the skin with a stratum corneum of 10-15 cell layers, ˜1 mm thick. The stratum corneum limits drug penetration to target receptors at the basal layer of the epidermis.
The proposed treatment is topical and repeated. An essential goal is to interrupt the itch-scratch cycle.
The preferred duration of action in the philtrum assay is >3 hr cooling, with another 3 hr residual cooling.
The formulation of FEMA 4557 should cool the philtrum for at least 3 hr and preferably longer, with residual cooling action.
In formulations with FEMA 4557, anti-inflammatory steroids, hormones, or emollients can be mixed.
The drug should be potent, cost-effective, and receptor-selective to avoid side effects.
The formulation for delivery should be easy to prepare.
The technology should be original and allow patentability for development.
The quality of sensation should be refreshingly cool or cold, without irritation or pain.
Identifying the ideal chemical class and molecule is a matter of continued and persistent experiment. When the criteria defined above, 95+% of known cooling molecules will fail! However, one N-alkyl, 2,2 diisopropyl butanamide structure, e.g., FEMA 4557, meets these criteria of successful implementation.

### FEMA 4557 Treatment of Nasal Cavity Inflammation

Another site for testing the acute anti-inflammatory actions of FEMA 4557 is the inflamed nasal mucosa. Inflammation of the nasal cavity is called rhinitis and has various degrees of severity. A simple runny nose, with watery discharge is inconsequential, except for a bit of sniffling. But when the condition is severe there is thick, purulent, and discolored discharge, accompanied by the subjective feeling of impaired airflow, loss of patency, and uncomfortable breathing. This is a serious form of nasal inflammation.

Inflammation in the nasal passages occurs when the mucosae overreact to allergens, viral or bacterial infections, inhaled irritants such as odors, perfumes, and smoke, changes in temperature or humidity, hormonal changes, or occupational exposure to chemicals, dust, or fumes. When anti-inflammatory steroids and antihistamines fail to control symptoms, surgery is the next alternative. Endoscopic sinus surgery is a procedure to remove obstructions, inflamed tissues, and nasal polyps. More recently, rhinologists have been using cryosurgical and electrolytic procedures to remove the nerves of the nasal cavity to treat recalcitrant rhinitis.

In this discovery, a specialized nebulizer with an electronically controlled compressor was used to deliver FEMA 4557 to the nasal cavity of a subject with rhinitis. A typical dosage regimen was a 1.0 to 1.5% FEMA 4557 wt/vol solution (10 to 15 mg/mL) over a 3 to 5 sec delivery period. A dosage schedule of two to three activation times per day for three to five days relieved symptoms and allows the subject to manage and control the rhinitis.

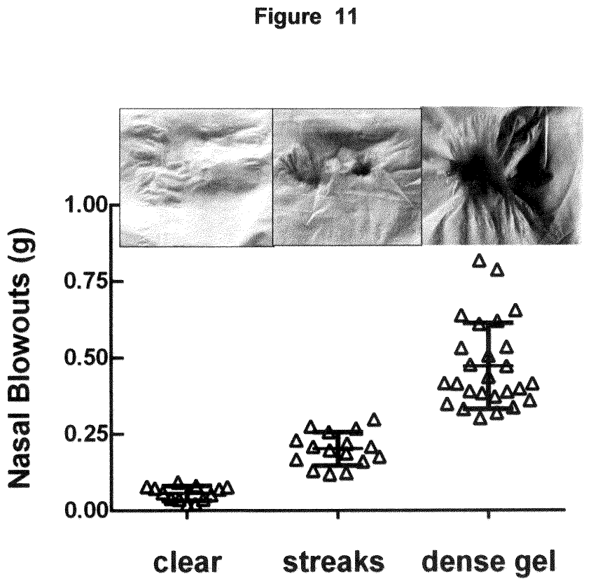

To establish baselines for measuring nasal inflammation, subjects were instructed to blow into a pre-weighed paper tissue and record the weight of the tissue after blowing. FIG. 11 shows that the weight of the “blow-outs” can be categorized as clear, streaks, or dense gel. The relative unit weights (mean±s.e.m.) were clear, 0.06±0.01 g, streaks 0.20±0.01 g, and dense gel 0.47±0.03 g. The weight of the blowouts provides an objective index of nasal cavity inflammation.

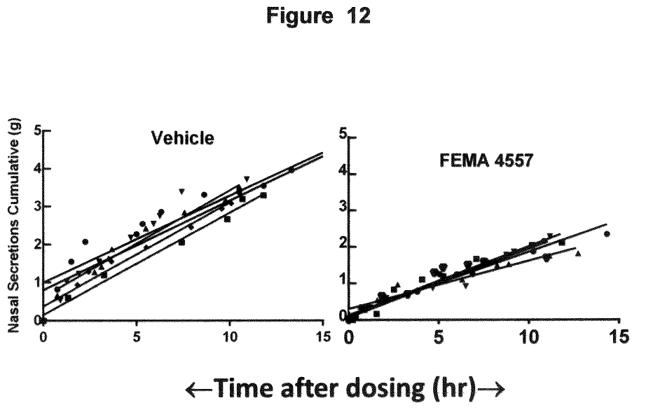

FIG. 12 shows the inhibition of nasal secretions in a rhinitis patient by topical administration of FEMA 4557, dissolved at 15 mg/mL in an ethanol, polysorbate 80, propanediol mixture. The test substance was rinsed into the nasal cavity with a Nasoneb® apparatus for 5 sec at t=0, 2, and 5 hr. The data points were fitted by linear regression (GraphPad Prism) and the 10-hr secretion measured as (avg.±s.e.m). The results were vehicle 3.16±0.10 g, and FEMA 4557 1.86±0.07 g, with n=6, 5 observations per group, respectively. The nasal secretion-10 hr unit for FEMA 4557 was significantly lower (P≤0.01) than vehicle when analyzed by the Kruskal-Wallis test for non-parametric data. The anti-inflammatory effect of FEMA 4557 on rhinitis is thereby demonstrated.

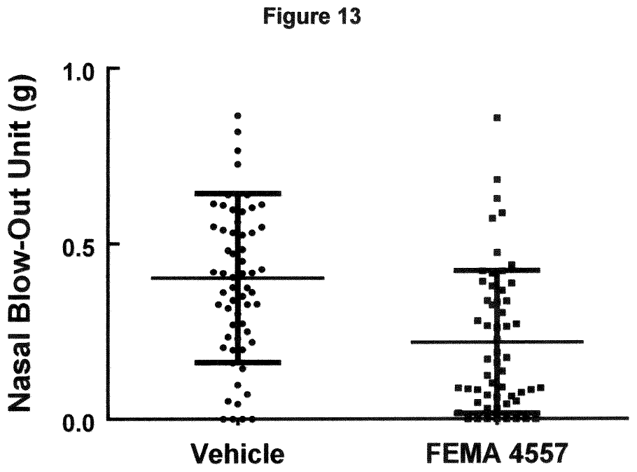

To further illustrate the inhibition of nasal secretions by topical administration of FEMA 4557, 15 mg/mL, the individual blowout units, in g per blowout, is shown in FIG. 13. FEMA 4557 was rinsed into the nasal cavity for 5 sec at t=0, 2, and 5 hr. The “blow-outs” from the nose were measured with a tissue paper and balance. The average±s.e.m. for blowouts were vehicle 0.38±0.03 g and FEMA 4557 0.21±0.03 g, with n=61, and n=56 observations per group, respectively. The blowout units were significantly different (P≤0.0001) when analyzed by the Kruskal-Wallis test for non-parametric data. Thus, the anti-inflammatory effect of FEMA 4557 is confirmed.

The nasal cavity mucosa is a complex system. When there is inflammation (rhinitis), a number of components are activated and sentient. Excess mucus secretion is the most dangerous pathological feature of rhinitis. Mucus is a transparent white protein when secreted but when mixed with exudates and cell debris it adopts a slimy, viscous yellow gel called phlegm or gunk. When copious, it is dense and unpleasant to look at when blown out onto a wipe (FIG. 11). When phlegm dries, it becomes crusted and hardened, and called “snot.” Phlegm can physically reduce nasal cavity volume, but the patient with rhinitis typically clears the mucus by blowing the nose or using irrigation with water or saline. Excess phlegm gives the opacity that appears in a CT scan and the diagnosis of severe inflammation. The weight of phlegm blown out onto a tissue paper provides a quantitative and dynamic index of nasal inflammation.

Formulations for nebulization with NasoNeb® used a standard recipe of 1 to 1.5% of FEMA 4557 (10 to 15 mg/mL), 2% Tween 80 (Polysorbate 80), 1 to 5% ethanol, and made up to 100% with water. The mixture is centrifuged in a FlackTek, Inc., Speedmixer for at least 10 min at 3000 rpm. The Speedmixer spins the sample in 3 dimensions. The result is a white homogeneous liquid suspension with some of the solids now dissolved. For dosing, ˜2 to 3 mL is loaded into the canister of the nebulizer. The nebulizer nozzle tip is placed in the nostril and activated by pressing a button for 3 to 5 sec using a clock timer. The delivered volume for a 3-sec and 5-sec activation resulted in 0.62±0.1 mL and 0.73±0.1 mL delivered per nostril, respectively. The nasal cavity is relatively tolerant to exogenous agents with irritant properties, as many rinses are used for rhinitis and sinusitis. For inflamed nasal passages, a rapid and efficient medication to reduce phlegm production can revolutionize rhinitis treatment. A simple 3- to 5-day course of nebulized FEMA 4557 to treat phlegm secretion, the worst consequence of rhinitis, may help save resources for managing rhinitis. This type of disorder affects up to 15% of the general population. The results herein suggest that rhinitis may be treatable as an acute inflammatory disease of the nasal mucosa, much like a mosquito bite or a pustule. When phlegm production is reduced to below 0.25 g per blowout event, the subject's nasal mucosa begins to recover and resurrects to clean and refreshed breathing. This resurrection can become permanent and maintained. This change may occur within three to five days after treatment. Thus, treatment of the inflammation of acute rhinitis with FEMA 4557 may halt and reverse the course of the disease.

### Acute Anti-Inflammatory Actions of FEMA 4557

Acute inflammation is a dynamic clinical event that accompanies tissue injury. A new anti-inflammatory mechanism of action can potentially treat many indications. Physical cooling by lowering tissue temperatures of injury relieves pain, but its benefits are not sustainable because cooling will cause cell damage by interference with metabolism. Chemical monoterpenoid cooling agents, such as I-menthol, are irritating and not considered anti-inflammatory. Here, we show FEMA 4557, the preferred embodiment, counteracts the acute inflammation caused by insect stings, heat, allergy, urticaria, and thermal injury. Furthermore, inflammation of the nasal membranes is curtailed.

The preferred embodiment of this discovery, FEMA 4557 is a non-selective inhibitor of a variety of inflammatory stimuli. The exact mechanisms of the anti-inflammatory effect of FEMA 4557 are not unknown. The presence of TRP channels on neuronal membranes hints at the inhibition of neurogenic inflammation as a possible mechanism. One intriguing possibility is that TRP activation inhibits the generation of negative interstitial fluid pressure (Pif) in the extracellular matrix. Vascular leakage occurs when fluids pass from the blood compartment into the extracellular matrix. Inflammatory mediators such as histamine open gaps or endothelial pores (Baluk et al. 1998). A negative Pif in the extracellular matrix creates suction pressure on fluids from the blood compartment, resulting in edema and the disruption of tissue architecture (Wiig, 2011). In previous studies with hormones and peptides, the applicant has correlated negative Pif to tissue edema in experimental studies (Gjerde et al., 1998; Gjerde et al., 2000). It is likely that FEMA 4557 acts on Pif. FEMA 4557 may be a prototype of an agent to treat acute inflammation.

### REFERENCES

1. Baluk P, Fine N W, Thomas H A, Wei E T, Mcdonald D M. Anti-inflammatory mystixin peptides inhibit plasma leakage without blocking endothelial gap formation. J Pharmacol Exp Ther. 1998; 284(2).
2. Behrendt, H-J et al., Characterization of the mouse cold-menthol receptor TRPM8 and vanilloid receptor type-1 VR1 using a fluorometric imaging plate reader (FLIPR) assay. Brit. J. Pharmacol. (2004) 1-9.
3. Bödding M, et al., Characterisation of TRPM8 as a pharmacophore receptor. Cell Calcium. 2007; 42(6):618-28.
4. Chen J, Guan S M, Sun W, Fu H. Melittin, the Major Pain-Producing Substance of Bee Venom. Neurosci Bull. 2016; 32(3):265-72.
5. Erman, M B New development in physiological cooling agents. Perfumer and Flavorist Vol. 32, 2007.
6. Feldberg W. Memoirs of the Fellows of the Royal Society. John Henry Gaddum. 1900-1965; 1967; (November):56-77.
7. Gjerde E A B, Woie K, Wei E T and Reed R K. Corticotropin-releasing hormone inhibits lowering of interstitial pressure in rat trachea after neurogenic inflammation. Eur. J. Pharmacol. 352:99-102, 1998.
8. Gjerde, E A, Wei, E T, Reed, R K, Lowering of interstitial fluid pressure after neurogenic inflammation is inhibited by mystixin-7 peptide, Am. J. Physiol. Heart Circ. Physiol, 279: H1377-H1382, 2000.
9. Johnson S et al. Trigeminal Receptor Study of High-Intensity Cooling Agents. J Agric Food Chem. 2018; 66(10):2319-23.
10. Jung M J, Wei E T. et al. A randomized, vehicle-controlled clinical trial of a synthetic TRPM8 agonist (DIPA-1-7) gel for itch. J Am Acad Dermatol. 2021; 84(3):869-71.
11. Kang S Y, Wei E T, et al. TRPM8 agonist (cryosim-1) gel for scalp itch: A randomized, vehicle controlled clinical trial. J Eur Acad Dermatology Venereol. 2022; 1-2.
12. Leffingwell J, Rowsell D. Wilkinson Sword cooling compounds: from the beginning to now. Perfume & Flavorist 39:34-44, 2014.
13. Leffingwell, J. C. Cooling ingredients and their mechanism of action. Handbook of cosmetic science and technology. Chpt. 65. Pp. 661-675. Barel, Paye, and Maibach. (edited). Informa Health Care, New York, 2009.
14. McKemy D D, Neuhausser W M, Julius D. Identification of a cold receptor reveals a general role for TRP channels in thermosensation. Nature. 2002; 416(6876):52-8.
15. Robert S M et al. New drugs on the horizon for cerebral edema: What's in the clinical development pipeline? Expert Opin Investig Drugs. 29:1099-1105, 2020.
16. Roswell D G et al. Acyclic carboxamides having a physiological cooling effect. U.S. Pat. No. 4,296,255 Oct. 20, 1981.
17. Sherkheli M A et al. Characterization of Selective TRPM8 Ligands and their Structure. J. Pharmacy and Pharmaceutical Sciences 2010; 13(2):242-53.
18. Smith R L et al. GRAS flavoring substances 24. Food Technol. 2009; 63(6):46-62.
19. Sun H S. Compounds with physiological cooling effect. U.S. Pat. No. 7,030,273 (2006).
20. Wei E T. Cool Esthesia airway hygiene: Stop stuffiness, cough and clear phlegm. Open Access Gov. 2023; 38(1):110-1.
21. Wei E T. Sensory/Cooling Agents for Skin Discomfort. Journal of Skin Barrier Research. 2012:14(2), 5-12.
22. Wei, E T. 1,2,3,6-Tetrahydropyrimidine-2-one compositions and therapeutic methods therewith for pain and inflammation. U.S. Pat. No. 6,743,801. Jun. 1, 2004.
23. Wei, E T. [((1R,2S,5R)-2-Isopropyl-5-methyl-cyclohexanecarbonyl)-amino]-acetic acid isopropyl ester and related compounds and their use in therapy. U.S. Pat. No. 8,426,463. Apr. 23, 2013.
24. Wei, E T. N-Alkylcarbonyl-amino acid ester compounds and their use for skin irritation, itch and pain. U.S. Pat. No. 6,599,524. Oct. 7, 2014.
25. Wei E T et al. Peripheral anti-inflammatory actions of corticotropin-releasing factor. Ciba Found Symp. 172:258-68, 1993; discussion 268-76.
26. Wei E T. and Thomas H A. Anti-Inflammatory Peptide Agonists. Annual Review of Pharmacology and Toxicology 33:91-108, 1993.
27. Widdicombe J H, Wine J J. Airway gland structure and function. Physiol Rev. 2015; 95(4):1241-319.
28. Wiig H. Pathophysiology of tissue fluid accumulation in inflammation. J Physiol. 2011; 589(12): 2945-53.
29. Wu Z H et al., Acute and subacute inhalation toxicity assessment of WS-23 in Sprague-Dawley rats. J Appl Toxicol. 2021; 41(11):1826-38.
30. Xu et al. Molecular mechanisms underlying menthol binding and activation of TRPM8 ion channel. Nat Commun. 2020. http://dx.doi.org/10.1038/s41467-020-17582-x)
31. Yogeswaran S et al. The role of Synthetic Coolants; WS-3 and WS-23, in modulating E-cigarette-induced Reactive Oxygen Species (ROS) in lung epithelial cells. Toxicol Reports [Internet]. 2022: 9(June): 1700-9. https://doi.org/10.1016/j.toxrep.2022.08.007

1. A method for treating inflammation of the skin or nasal mucosa of a mammal in need of treatment thereof, comprising:
providing a liquid or semi-liquid formulation of a therapeutically effective amount of N-ethyl-2,2-diisopropylbutanamide, a compound having the structure of Formula 1 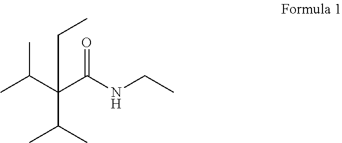
and; topically applying said formulation to the skin or nasal mucosa of the mammal.

2. The method as in claim 1 wherein the therapeutically effective amount of Formula 1 on the skin is from about 50 to 150 mg per dose.

3. The method as in claim 1 wherein the formulation for the skin has from about 8% to 32% by weight of the Formula 1 compound.

4. The method as in claim 1 wherein the inflammation of the skin being treated is dermatitis.

5. The method as in claim 1 wherein the inflammation of the nasal mucosa being treated is rhinitis.

6. The method as in claim 1 wherein the treatment of inflammation is the treatment of the reactions of vascularized tissues to localized injury.

7. The method as in claim 1 wherein the treatment of inflammation is treatment of swelling, redness, and loss of function of living tissues.

8. The method as in claim 1 wherein the inflammation on the skin being treated are the symptoms of irritation, itch, soreness, burning sensations, and pain.

9. The method as in claim 4 wherein the dermatitis is caused by diabetes mellitus.

10. The method as in claim 4 wherein the dermatitis is caused by thermal injury.

11. The method as in claim 4 wherein the dermatitis is caused by skin allergy.

12. The method as in claim 4 wherein the dermatitis in need of treatment is the inflammatory response to insect bites.

13. The method as in claim 5 wherein the inflammation being treated are the symptoms and signs of excess nasal phlegm production.

14. A method for treating inflammation of the skin or nasal mucosa of a mammal in need of treatment thereof, comprising:
a liquid or semi-liquid formulation having a therapeutically effective amount of a compound of Formula 2, 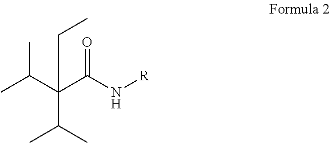
wherein R is n-propyl, cyclopropyl, or acetyl
and; topically applying said formulation to the skin or nasal mucosa of the mammal.

15. The method as in claim 14 wherein the Formula 2 compound is N-isopropyl-2,2-diisopropylbutanamide.

16. The method as in claim 14 wherein the Formula 2 compound is N-cyclopropyl-2,2-diisopropylbutanamide.

17. The method as in claim 14 wherein the Formula 2 compounds is N-acetyl-2,2-diisopropylbutanamide.

18. The method as in claim 14 wherein the therapeutically effective amount of Formula 1 on the skin is from about 50 to 150 mg per dose.

19. The method as in claim 14 wherein the formulation for the skin has from about 8% to 32% by weight of the Formula 1 compound.

20. The method as in claim 14 wherein the inflammation of the skin being treated is dermatitis.

21. The method as in claim 14 wherein the inflammation of the nasal mucosa being treated is rhinitis.

22. A method for treating inflammation of the nasal mucosa of a mammal in need of treatment thereof, comprising topical administration of N-ethyl-2,2-diisopropylbutanamide.

23. The method as in claim 22 wherein the administered concentration of N-ethyl-2,2-diisopropylbutanamide is 5 to 15 mg/mL and the administered dose is 2.5 mg to 7.5 mg per nostril.
